(19) United States
(12) Patent Application Publication
(10) Pub. No.: US 2012/0158137 A1
(43) Pub. Date: Jun. 21, 2012

(54) PEEK-RICH BONE SCREW

(76) Inventor: Leo Arieh Pinczewski, New South Wales (AU)

(21) Appl. No.: 13/327,727

(22) Filed: Dec. 15, 2011

Related U.S. Application Data
(60) Provisional application No. 61/423,566, filed on Dec. 15, 2010.

(51) Int. Cl. A61F 2/08

(52) U.S. Cl. 623/13.14

(57) ABSTRACT

A surgical screw for anchoring a flexible member to a bone. The screw may include a primary thread having a continuous helical ridge that curves around the shaft of the screw. A set of secondary threads may be disposed on, and follow, the continuous helical ridge around the screw. The set of secondary threads may have the same periodicity as the primary thread. The primary thread may comprise both a sharp portion and a blunt portion to prevent damage to the flexible member.

### CROSS-REFERENCE TO RELATED APPLICATIONS

This application claims the benefit of U.S. Provisional Application No. 61/423,566, filed Dec. 15, 2010 which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not Applicable.

### BACKGROUND

1. The Field of the Present Disclosure

The present disclosure relates generally to surgical implants, and more particularly, but not necessarily entirely, to surgical screws for anchoring flexible members, such as grafts, tendons, ligaments and implants, to bones during surgery.

2. Description of Related Art

Surgical screws are increasingly employed by orthopedic surgeons in the repair of injuries. In particular, surgical screws may be utilized to anchor flexible members, such as grafts, tendons, ligaments, or implants to a bone. In a typical surgery, a bore is first prepared by the surgeon in the bone. A free end of the flexible member may then be inserted into the bore by the surgeon. The surgeon may then install a surgical screw into the bore. The surgical screw may include threads that engage the sidewall of the bore in order to secure the flexible member in the bore. The surgical screw may secure the flexible member to the bone by clamping the flexible member against the sidewall of the bore.

In some cases, the high tension imposed on the currently available bone screws causes the flexible member to be damaged. In other cases, the flexible member is caused to pull out while the surgical screw remains secured in the bore, due to an inadequately weak engagement between the screw and the flexible member.

In still further cases, the high tension on the flexible member may cause the surgical screw itself to pullout of the bore and release the flexible member.

In an attempt to reduce those problems, some previously available surgical screws have been designed with enhanced engagement features, such as larger and sharper threads that provide a more secure engagement with the sidewalls of the bore in the bone. However, the use of larger and sharper threads may inadvertently cause the flexible member to fail as the threads may sever or weaken the flexible member. Attempts to minimize damage to the flexible member while improving the engagement between surgical screws and the bone have been attempted, but with limited success.

One attempt to avoid graft or tendon damage is disclosed in U.S. Pat. No. 5,383,878 (hereinafter “the '878 patent”), which issued on Jan. 24, 1995 to applicant, however, the risk of pullout of the flexible member is increased. The '878 patent discloses a surgical screw having a shank with a thread formed along the shank. The thread formed along the shank is devoid of an outermost cutting line. In particular, the thread on the shank has a smooth or soft thread so as to provide an interlocking fixation of a bone end of a tendon graft within a prepared hole of a bone. Stated another way, the threads of the surgical screw of the '878 patent have no outermost cutting line which would normally helically follow the thread crest. The surgical screw of the '878 patent was an improvement over the previously available surgical screws in that its threads did not cut into, and thereby damage, the graft during implantation. Although the surgical screw of the '878 patent did succeed in minimizing damage to a graft caused by “sharp” threads, the soft threads of the surgical screw of the '878 patent may not always avoid graft or tendon pullout, or pullouts of the screw from the bone. For example, the use of the “soft threads” may cause the surgical screw to retreat from the bone hole after implantation.

Another attempt to avoid graft or tendon damage is disclosed in U.S. Pat. No. 6,589,245 (hereinafter “the '245 patent”), which issued on Jul. 8, 2003 to Weiler et al. The '245 patent discloses an interference screw having a sharp threading disposed adjacent the penetrating end and a blunt threading in the following region. The purported purpose of the blunt threading is to ensure that transplant tissue is not severed or separated in the region of the blunt threading. While an improvement, the reduced pressure or force imposed by the interference screw disclosed in the '245 patent on the graft or tendon, presents an increased risk in failing to prevent graft or tendon pull out, and the screw itself is at risk of pulling out of a bore.

The prior art is thus characterized by several disadvantages that are addressed by the present disclosure. The present disclosure minimizes, and in some aspects eliminates, the above-mentioned failures, and other problems, by utilizing the methods and structural features described herein.

It appears to applicant that the point of all other bone screws is to increase their hold on bone. In contrast, one key point of the present disclosure is to increase the hold on the flexible member (e.g. on the graft or tendon) and prevent pullout of the flexible member from the bone.

In applicant's experience, the bone screw may be retained in the bore, even though the tendon for which it is responsible has slipped out of the bore. Surgeons are generally concerned with keeping the screw in the bone, but in fact, the point of the surgery should actually be to keep the tendon (flexible member) in its position in the bone. The '878 patent discloses a soft thread screw whose primary aim to avoid damage to the flexible member, while in contract, one key purpose of the present disclosure is to enhance the pullout force required to dislodge the flexible member. That purpose is not addressed by either the '245 patent or the '878 patent. The present disclosure discloses a new and novel screw that keeps the flexible member in position in the bone, while also increasing the hold of the screw itself to the bone, minimizing damage to the flexible member, and yet allows easier removable of the screw from the bone without further damage to surrounding tissue or bone.

The features and advantages of the present disclosure will be set forth in the description which follows, and in part will be apparent from the description, or may be learned by the practice of the present disclosure without undue experimentation. The features and advantages of the present disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a side view of a surgical screw pursuant to an embodiment of the present disclosure;

FIG. 2 is a cross-sectional, side view of the surgical screw shown in FIG. 1;

FIG. 3 is a perspective view of the surgical screw shown in FIG. 1;

FIG. 4 is a perspective view of the surgical screw shown in FIG. 1;

FIG. 5 is a side view of a surgical screw pursuant to an embodiment of the present disclosure;

FIG. 6 is a cross-sectional, side view of the surgical screw shown in FIG. 5;

FIG. 7 is a perspective view of a surgical screw pursuant to an embodiment of the present disclosure;

FIG. 8 is a perspective view of the surgical screw shown in FIG. 7;

FIG. 9 is a side view of the surgical screw shown in FIG. 7;

FIG. 10 is a cross-sectional side view of the surgical screw shown in FIG. 7;

FIG. 11 is a side view of a surgical screw pursuant to an embodiment of the present disclosure;

FIG. 12 is a side view of a surgical screw pursuant to an embodiment of the present disclosure; and

FIGS. 13 and 14 depict a method of anchoring a flexible member to a bone pursuant to an embodiment of the present disclosure.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

Applicant has discovered an improved surgical screw, or interference device, that minimizes thread damage to flexible members, reduces the risk of flexible member pullout, reduces the risk of screw pullout, and allows easier removal of the screw when needed in the future without further damage to surrounding tissue or bone. Applicant's surgical screw may include a dual action thread. For example, the threads of the Applicant's surgical screw may have a form factor that varies along the length of the screw. The threads near the distal end of the screw may have a sharp cutting line for improved engagement with a sidewall of a bore in a bone while the threads toward the proximal end may have a “smooth” cutting line to reduce damage to grafts.

Applicant's surgical screw may further comprise a primary thread and a set of secondary threads. The set of secondary threads may be disposed on, and follow, the primary thread as it curls around the shaft of the surgical screw. A height or amplitude of the primary thread may be significantly larger than the height or amplitude of the set of secondary threads. The primary thread and the set of secondary threads may be coextensive along the shaft of the surgical screw.

Applicant's surgical screw may operate to decrease the risk of the secured flexible member pulling out of the bone, while the surgical screw remains fixed in the bore. Applicant's surgical screw may further decrease incidences of slippage between the flexible member and the surgical screw. Applicant's surgical screw is also designed to avoid pullout of the screw itself, from the bone. Thus, it will be appreciated that Applicant's surgical screw may increase the hold on the flexible member and prevent pullout of the flexible member, or the screw itself, from the bone.

Applicant's surgical screw may include a macro-shape that engages the bone and also a micro-engagement feature to prevent the flexible member from being pulled from a bore while the surgical screw remains in place. The nature of the micro-engagement feature of the present disclosure is unique in that it enhances resistance to the pullout of the flexible member, without comprising the surgical screw's ability to be inserted or extracted.

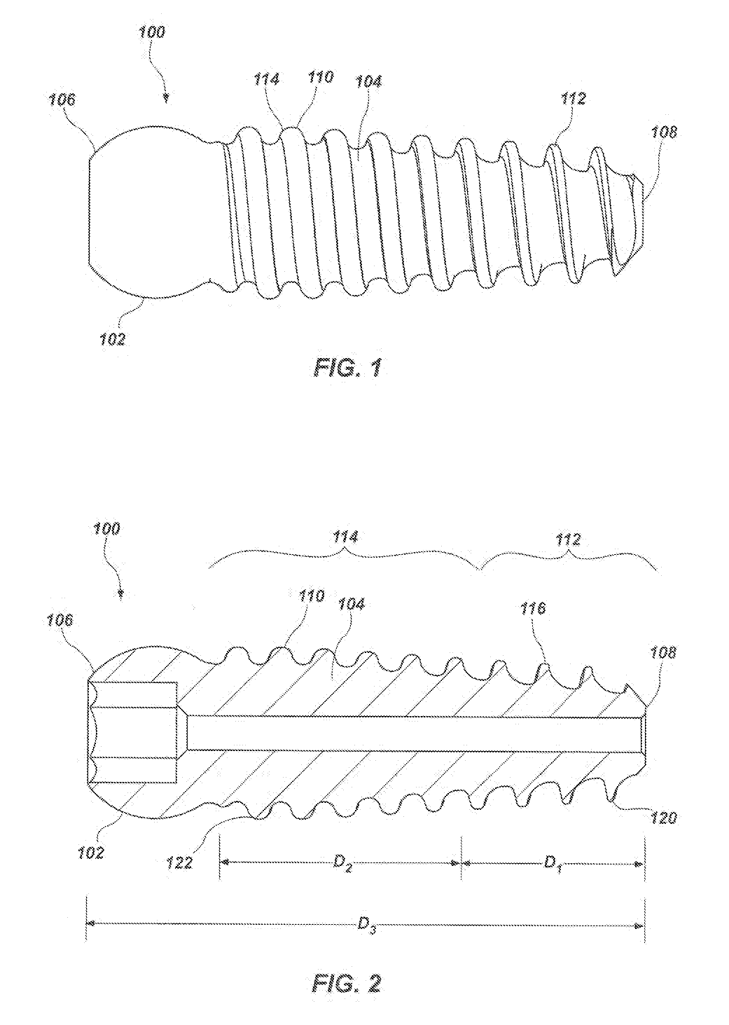

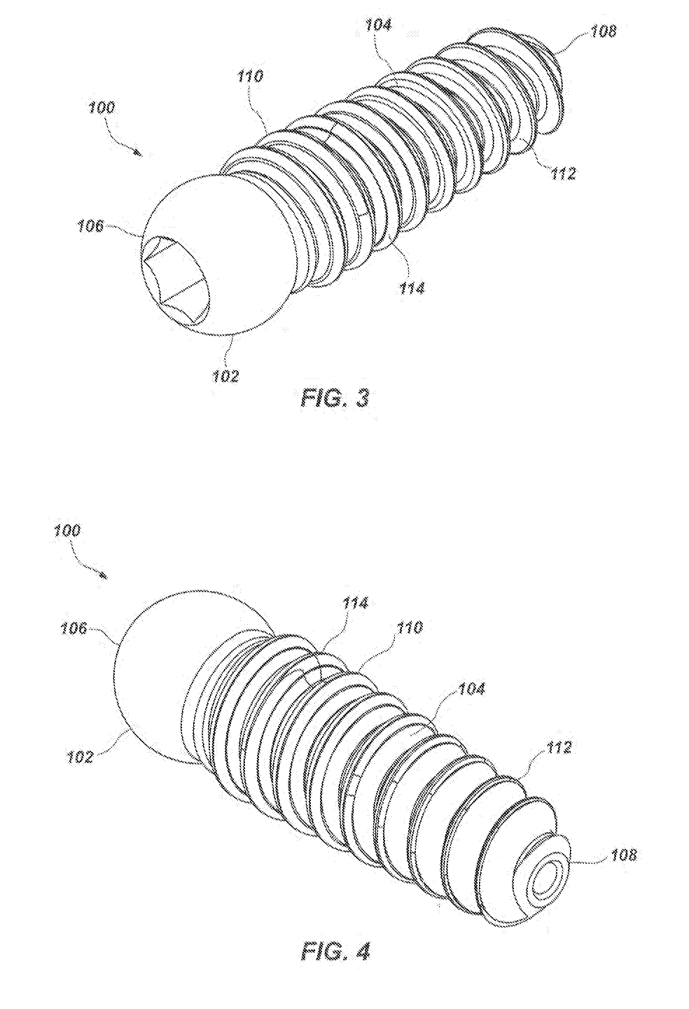

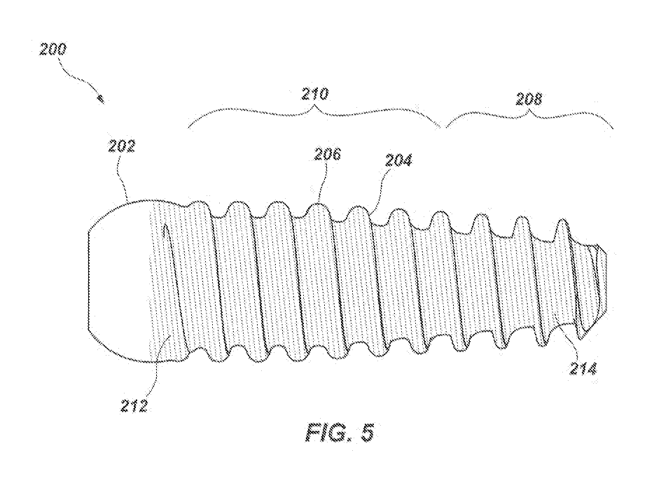

Referring now to FIGS. 1-4, there is depicted a surgical screw 100 pursuant to an embodiment of the present disclosure. In an embodiment of the present disclosure, the screw 100 may be a tapered screw as shown. The screw 100 may comprise a screw head 102 and a shaft member 104. The screw 100 may extend from a proximal end 106 to a distal end 108. The screw head 102 may be located at the proximal end 106. The shaft member 104 itself may extend from the screw head 102 to the distal end 108 of the screw 100. The shaft member 104 of the screw 100 may comprise threads 110. As will now be described, the thread form of the threads 110 may vary along the length of the shaft member 104, meaning, the shape, path and physical space taken up by the threads 110 may vary along the length of the shaft member 104. Stated another way, the thread 110 may vary in size, thickness, shape, spacing, or in any suitable physical manner.

The threads 110 may comprise a first thread portion 112 and a second thread portion 114. The first thread portion 112 may extend a distance of D₁ from the distal end 108 of the screw 100 toward the proximal end 106 of the screw 100 as shown in FIG. 2. The second thread portion 114 may extend a distance of D₂ from the end of the first thread portion 112 toward the proximal end 106 of the screw 100 as also shown in FIG. 2. The second thread portion 114 may terminate at the head 102 of the screw 100.

As perhaps best seen in FIG. 2, but visible in the other figures as well, the surface topography or thread form of the threads 110 varies along the shaft member 104 of the screw 100. In particular, the threads 110 in the first thread portion 112 may comprise a sharper cutting line 116 than the threads 110 in the second thread portion 114. The threads 110 in the second thread portion 114 may be more rounded than the threads 110 in the first thread portion 112. The threads 110 in the second thread portion 114 may comprise a more sinusoidal shape than the threads 110 in the first thread portion 112. In an embodiment of the present disclosure, the threads 110 in the first thread portion 112 may be more v-shaped than the threads 110 in the second thread portion 114.

In an embodiment of the present disclosure, the threads 110 in the first thread portion 112 may have a sharper thread crest 120 than a thread crest 122 of the threads 110 in the second portion 114. In an embodiment of the present disclosure, the threads 110 in the first thread portion 112 may have a stronger bite into the bone than the threads 110 in the second portion 114. In an embodiment of the present disclosure, the threads 110 in the first thread portion 112 may form a higher pressure between the thread crest 120 and the bone (not shown) than the thread crest 122 of the threads 110 in the second portion 114. In an embodiment of the present disclosure, the threads 110 in the first thread portion 112 may have a higher-pressure thread form while the threads 110 in the second portion 114 may have a lower-pressure thread form. (A sharper thread concentrates a screw force into a smaller area resulting in a higher force per unit area and thus a higher pressure.) In an embodiment of the present disclosure, the thread form along the shaft member 104 of the screw 100 varies along its length, with the sharpness of the threads 110 decreasing as the threads 110 get closer to the proximal end 106.

In an embodiment of the present disclosure, the length, D₁, of the first thread portion 112 may be approximately 20-40% of the length D₃, of the screw 100, or the length D₁ may be approximately 40%-60% of the length, D₃, of the screw 100, depending on the embodiment. In an embodiment of the present disclosure, the length, D₂, of the second thread portion 114 may be approximately 50%-70% of the length, D₃, of the screw 100, or about 60%. In an embodiment of the present disclosure, the threads 110 in the second thread portion 114 may be fully round at their major diameter and extend into a similar full radius in the root diameter of the threads 110.

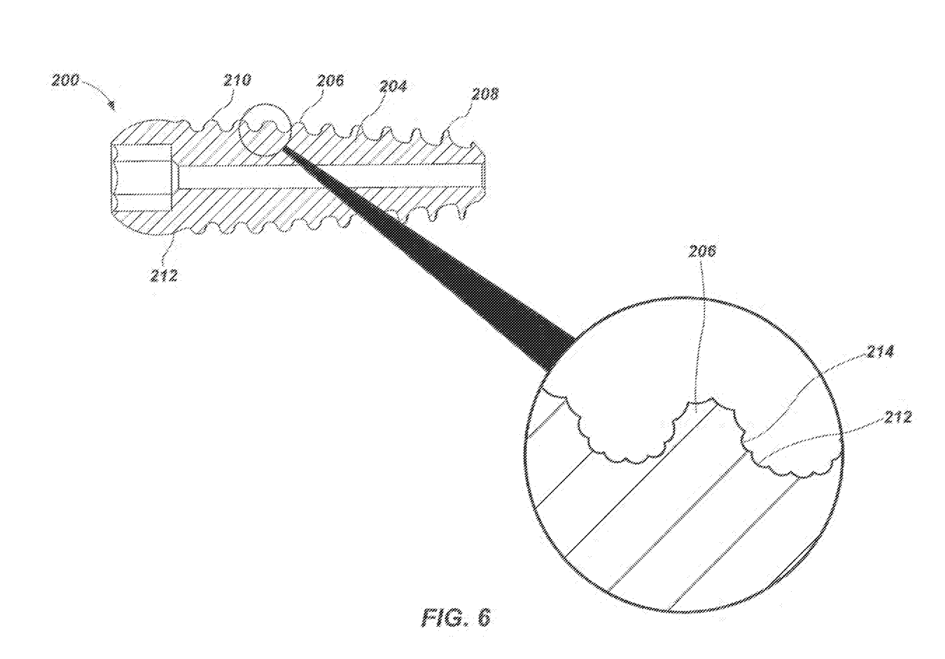

Referring now to FIGS. 5 and 6, there is shown a surgical screw 200 pursuant to an embodiment of the present disclosure. The screw 200 may have all of, some of, or none of the features of the screw 100 described in relation to FIGS. 1-4, above. As shown in FIGS. 5 and 6, the screw 200 may comprise a head 202 and a shaft member 204. The shaft member 204 may comprise threads 206. The shaft member 204 may include a first thread portion 208 and a second thread portion 210. As explained above, the threads 206 of the first thread portion 208 may have a sharper cutting line than the threads 206 of the second thread portion 210.

The screw 200 may further comprise a outer surface 212. Formed in the surface 212 may be a micro-texture 214. The micro-texture 214 may increase the coefficient of friction of the screw 200 to prevent slippage of a tendon or graft. The micro-texture 214 may be characterized by surface roughness parameters (per ISO definition) Ra 0.2-2.0 and Rz 4 to 20. The micro-texture 214 may be produced using the following machining parameters: depth of cut 0.1 mm to 0.3 mm, pitch 0.1 to 0.5 mm, and a cutting tip radius of 0.075 mm to 0.2 mm. The micro-texture 214 on the outer surface 212 may comprise serrations. The micro-texture 214 on the outer surface 212 may comprise asperities.

In an embodiment of the present disclosure, the screws 100 and 200 may be formed using an injection molding process. The screws 100 and 200 may be formed of PEEK.

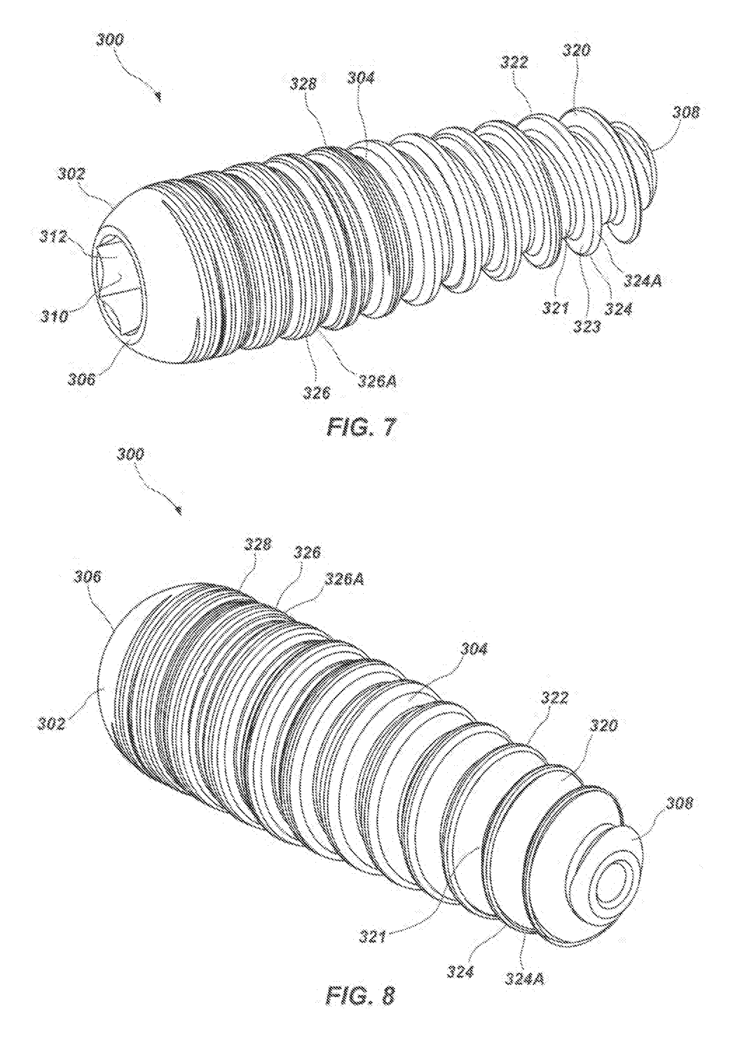

Referring now to FIGS. 7-10, there is depicted a surgical screw 300 according to an embodiment of the present disclosure. The screw 300 may comprise a screw head 302 and a shaft member 304. The screw 300 may extend from a proximal end 306 to a distal end 308. The screw head 302 may be located at the proximal end 306. The shaft member 304 may extend from the screw head 302 to the distal end 308 of the screw 300. Formed in the screw head 302 may be a recess or socket 310 having a surface 312 configured and adapted for receiving a drive shaft of a tool (not shown).

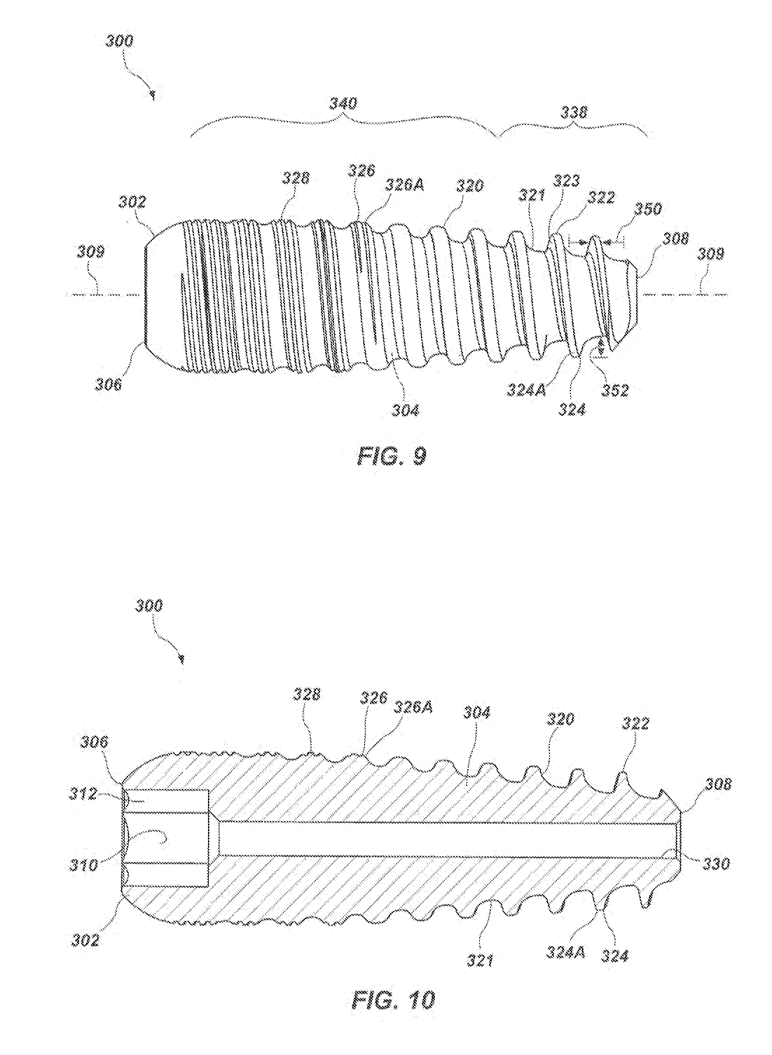

The screw 300 may comprise a primary thread 320 extending from the distal end 308 toward the proximal end 306. The primary thread 320 may comprise a continuous helical ridge 322 curling around the shaft member 304. The curls of the primary thread 320 may be separated by valleys 321, and the primary thread is further defined by sidewalls 323. A thread width of the primary thread 320, indicated by the double arrows marked with the reference numeral 350 in FIG. 9, may increase in the distal to proximal direction. A thread height or amplitude of the primary thread 320, indicated by the double arrows marked with the reference numeral 352 in FIG. 9, may decrease in the distal to proximal direction.

The primary thread 320 may comprise a first portion 324 and a second portion 326. The first portion 324 may be nearer the distal end 308, meaning, as part of a distal section 338. The second portion 326 may sequentially follow the first portion 324 and extend toward the proximal end 306, meaning, as part of a proximal section 340. A cutting edge 324A of the first portion 324 may be sharper than a cutting edge 326A of the second portion 326. The transition between the first portion 324 and the second portion 326 may be abrupt. Alternatively, the transition between the first portion 324 and the second portion 326 may be gradual.

Although part of the same primary thread 320, the form of the primary thread 320 in the first portion 324 and the second portion 326 may vary. The first portion 324 and the second portion 326 may together define a thread length of the primary thread 320. In an embodiment of the present disclosure, the first portion 326 may extend approximately 50% to 70% of the length of the primary thread 320. In an embodiment of the present disclosure, the second portion 326 may extend about 50% to 70% of the length of the primary thread 320, and may for example extend about 60% of that length.

A set of secondary threads 328 may also be disposed on the shaft member 304 of the screw 300. In an embodiment of the present disclosure, the set of secondary threads 328 may comprise one of a single thread or a plurality of threads. For example, the set of secondary threads 328 may comprise one thread, a pair of threads, three threads, four threads or more threads.

In an embodiment of the present disclosure, the set of secondary threads 328 may be disposed on, and follow, the continuous helical ridge 322 of the primary thread 320. In an embodiment of the present disclosure, the set of secondary threads 328 may be disposed on the peaks of the continuous helical ridge 322 of the primary thread 320. In an embodiment of the present disclosure, the set of secondary threads 328 may be either present or absent in the valleys 321 between the curls or peaks of the primary thread 320.

The set of secondary threads 328, may start on the primary thread 320, beginning on an upper portion of the shaft member 304 and extend toward the proximal end 306 of the screw 300. The set of secondary threads 328 may extend in the proximal direction closer to the proximal end 306 of the screw 300 than the primary thread 320. That is, the secondary threads 328 may extend beyond the primary thread 320 in the proximal direction on the shaft member 304. Conversely, the primary thread 320 may extend to the distal end 308 of the screw 300 while the secondary threads 328 may stop short of the distal end 308.

The primary thread 320 may have a periodicity, e.g., axial length per curl, meaning the axial distance along a longitudinal axis 309 of the screw 300 per revolution of the primary thread 320 as shown in FIG. 9. The set of secondary threads 328 may also have a periodicity. In an embodiment of the present disclosure, the periodicity of the primary thread 320 and of the set of the secondary threads 328 may be equal or substantially equal. The primary thread 320 may comprise a thread height and the set of secondary threads 328 may also comprise a thread height. In an embodiment of the present disclosure, the thread height of the set of secondary threads 328 may be 20% or less than the height of the primary thread 320. In an embodiment of the present disclosure, the thread height of the set of secondary threads 328 may be 10% or less than the height of the primary thread 320. As best seen in FIG. 10, the surgical screw 300 may include a hollow passageway or cannulation 330.

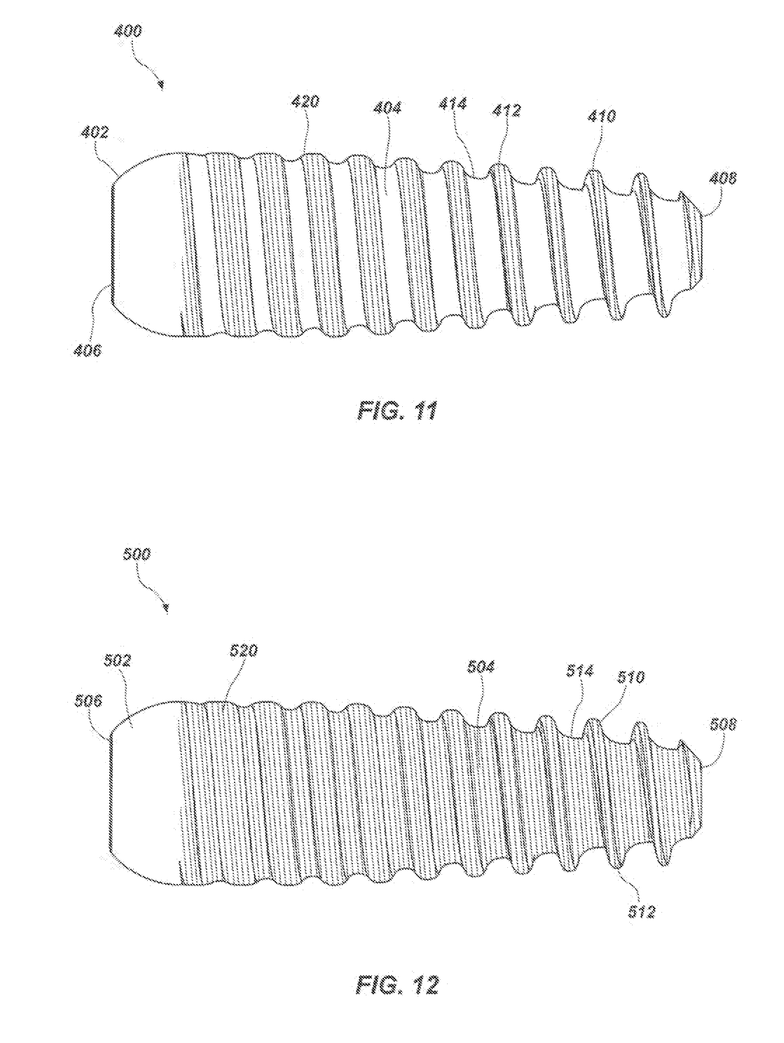

Referring now to FIG. 11, there is depicted a surgical screw 400 according to an embodiment of the present disclosure. The screw 400 may comprise a screw head 402 and a shaft member 404. The screw 400 may extend from a proximal end 406 to a distal end 408. The screw head 402 may be located at the proximal end 406. The shaft member 404 may extend from the screw head 402 to the distal end 408 of the screw 400.

The screw 400 may comprise a primary thread 410 extending from the distal end 408 toward the proximal end 406. The primary thread 410 may comprise a continuous helical ridge 412 curling around the shaft member 404. The curls of the primary thread 410 may be separated by valleys 414. The form of the primary thread 410 may be substantially the same as the primary thread 320 of the surgical screw 300 as described above.

A set of secondary threads 420 may also be disposed on the shaft member 404 of the screw 400. In an embodiment of the present disclosure, the set of secondary threads 420 may comprise one of a single thread or a plurality of threads. For example, the set of secondary threads 420 may comprise one thread, a pair of threads, three threads or four threads.

In an embodiment of the present disclosure, the set of secondary threads 420 may be disposed on, and follow, the continuous helical ridge 412 of the primary thread 410. In an embodiment of the present disclosure, the set of secondary threads 420 may be disposed on the peaks of the continuous helical ridge 412 of the primary thread 410. In an embodiment of the present disclosure, the set of secondary threads 420 may be absent in the valleys 414 between the curls of the primary thread 410.

In an embodiment of the present disclosure, the set of secondary threads 420 may extend from the distal end 408 of the surgical screw 400 toward the proximal end 406 of the surgical screw 400 following the continuous helical ridge 412 of the primary thread 410 and above the valleys 414. In an embodiment, the set of secondary threads 420 may be coextensive with the primary thread 410. In an embodiment, the set of secondary threads 420 may extend beyond the end of the primary thread 410 in the direction of the proximal end 406.

Referring now to FIG. 12, there is depicted a surgical screw 500 according to an embodiment of the present disclosure. The screw 500 may comprise a screw head 502 and a shaft member 504. The screw 500 may extend from a proximal end 506 to a distal end 508. The screw head 502 may be located at the proximal end 506. The shaft member 504 may extend from the screw head 502 to the distal end 508 of the screw 500.

The screw 500 may comprise a primary thread 510 extending from the distal end 508 toward the proximal end 506. The primary thread 510 may comprise a continuous helical ridge 512 curling around the shaft member 504. The curls of the primary thread 510 may be separated by valleys 514. The form of the primary thread 510 may be substantially the same as the primary thread 320 of the surgical screw 300 as described above.

A set of secondary threads 520 may also be disposed on the shaft member 504 of the screw 500. In an embodiment of the present disclosure, the set of secondary threads 520 may comprise one of a single thread or a plurality of threads. For example, the set of secondary threads 520 may comprise one thread, a pair of threads, three threads or four threads.

In an embodiment of the present disclosure, the set of secondary threads 520 may extend from the distal end 508 of the surgical screw 500 toward the proximal end 506 of the surgical screw 500 along the continuous helical ridge 512 of the primary thread 510 and in the valleys 514. In an embodiment, the set of secondary threads 520 may be coextensive with the primary thread 510. In an embodiment, the set of secondary threads 520 may extend beyond the end of the primary thread 510 in the direction of the proximal end 506.

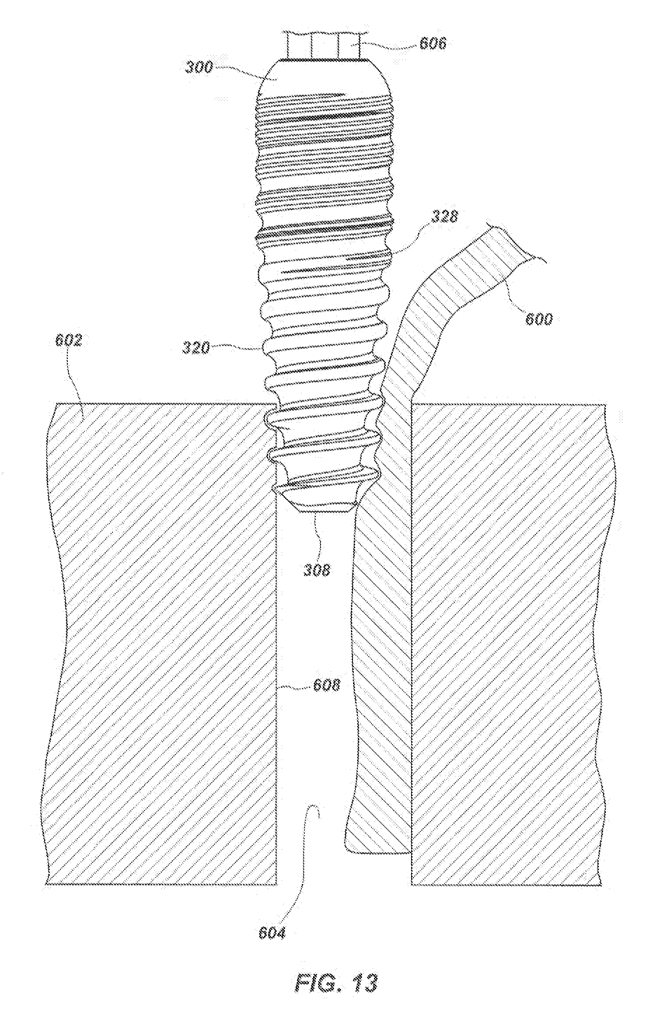

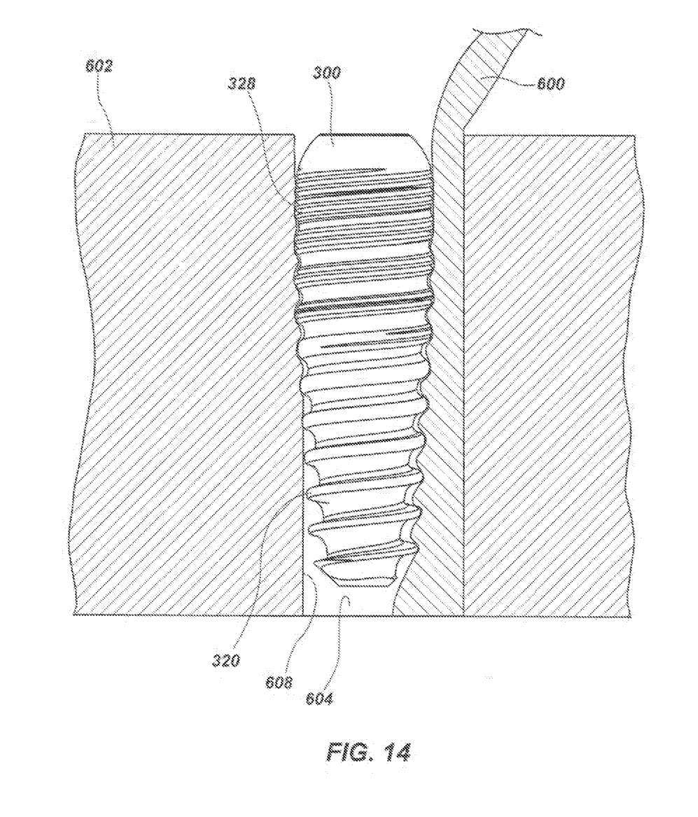

Referring now to FIGS. 13 and 14, there is depicted a process for anchoring a flexible member 600 to a bone 602 using the surgical screw 300. It will be appreciated that any one of the surgical screws disclosed herein may be used in place of the surgical screw 300. To begin, a bore 604 may be formed in the bone 602 as is known to one having ordinary skill in the art, using for example, a surgical drill. Once the bore 604 is formed, the flexible member 600 may be installed into the bore 604.

The surgical screw 300 may then be installed and advanced into the bore 604 using a tool 606 that rotates the screw 300. As the screw 300 is first advanced, the primary thread 320 near the distal end 308 may engage a sidewall 608 of the bore 604 and the flexible member 600. As the screw 300 is further advanced, the set of secondary threads 328 may engage the sidewall 608 of the bore 604 and the flexible member 600. When fully installed, as depicted in FIG. 14, both the primary thread 320 and the set of secondary threads 328 may engage the sidewall 608 of the bore 604 and the flexible member 600.

It will be appreciated that the dual engagement of both the primary thread 320 and the set of secondary threads 328 with the sidewall 608 of the bore 604, improves the ability of the screw 300 to resist pullout, and allows the screw 300 to be removed without further damaging any tissue or bone. The addition of the secondary threads 328 allows in particular for an increased hold in the flexible member 600, and a simultaneous increase in the hold of the screw 300 to the bone sidewalls 608, and yet without undue resistance to future removal of the screw 300: in short, simply unscrew the screw 300 to remove it. The set of secondary threads 328 may engage the sidewall 608 in the depression formed by the primary thread 320. It will thus be appreciated that the insertion torque and the removal torque are not affected much or at all, because the torque is a function of the threaded engagement between the screw 300 and the tissue. That is, while the screw 300 may be easily installed and removed using a tool, the screw 300 may also provide enhanced resistance to pullout, due to the dual engagement by both the primary thread and the secondary thread, yet still allow for easy removal by simply unscrewing the screw 300. The screw 300 thereby avoids further damage the flexible member 600 or the bone 602, during removal.

It will be appreciated that the structure and apparatus disclosed herein is merely one example of a means for preventing pullout of a surgical screw, and it should be appreciated that any structure, apparatus or system for preventing pull out of a surgical screw which performs functions the same as, or equivalent to, those disclosed herein are intended to fall within the scope of a means for preventing pull out of a surgical screw, including those structures, apparatus or systems for preventing pull out of a surgical screw which are presently known, or which may become available in the future. Anything which functions the same as, or equivalently to, a means for preventing pull out of a surgical screw falls within the scope of this element.

In accordance with the features and combinations described above, a useful method of fixating a flexible member includes the steps of:

(a) drilling a bore in a bone; and

(b) securing the flexible member in the bore using a surgical screw;

wherein the surgical screw comprises a head and a shaft member, the shaft member having a first thread portion and a second thread portion;

wherein the first thread portion has sharper threads as compared to threads of the second thread portion.

In accordance with the features and combinations described above, a useful method of fixating a flexible member includes the steps of:

(a) drilling a bore in a bone; and

(b) securing the flexible member in the bore using a surgical screw;

wherein the surgical screw comprises an outer surface;

wherein the outer surface comprises a micro-texture formed thereon.

In accordance with the features and combinations described above, a useful method of fixating a flexible member includes the steps of:

(a) drilling a bore in a bone; and

(b) securing the flexible member in the bore using a surgical screw;

wherein the surgical screw comprises an outer surface;

wherein the outer surface comprises a primary thread and a set of secondary threads.

An embodiment of the present disclosure may include a surgical screw having a shaft member with a longitudinal axis, a first thread portion extending along a first length of the shaft member, and a second thread portion extending along a second length of the shaft member, wherein the first thread portion has a cutting edge that is sharper than the second thread portion.

An embodiment of the present disclosure may include a surgical screw having a shaft member with a longitudinal axis, a thread extending along a length of the shaft member, wherein the surface topography or thread form of the thread varies along the length of the shaft member.

An embodiment of the present disclosure may include a surgical screw having a shaft member with a longitudinal axis, wherein said shaft member comprises a dual action thread.

An embodiment of the present disclosure may include a surgical screw having a shaft member with a longitudinal axis, wherein said shaft member comprises a thread, said thread having a non-uniform thread form.

An embodiment of the present disclosure may include a surgical screw having a shaft member with a longitudinal axis, wherein said shaft member comprises a varied thread.

An embodiment of the present disclosure may include a surgical screw having a shaft member with a longitudinal axis, wherein said shaft member comprises a distal end and a proximal end, a thread extending from the distal end toward the proximal end, wherein the thread is sharper at the distal end of the shaft member.

An embodiment of the present disclosure may include a surgical screw having a shaft member with a longitudinal axis, wherein said shaft member comprises a distal end and a proximal end, a thread extending from the distal end toward the proximal end, wherein the thread form varies along the shaft member of the screw.

An embodiment of the present disclosure may include a surgical screw having a shaft member with a longitudinal axis, wherein said shaft member comprises a distal end and a proximal end, a thread extending from the distal end toward the proximal end, wherein the thread forms a high pressure at the distal end and a lower pressure at the proximal end when the shaft member of the screw is installed in a bore in a bone.

An embodiment of the present disclosure may include a surgical screw having a shaft member with a longitudinal axis, wherein said shaft member comprises threads that engage a bore wall with a high pressure near the distal end of the screw and a low pressure toward the proximal end of the screw.

An embodiment of the present disclosure may include a surgical screw having a shaft member with a longitudinal axis, wherein said shaft member comprises threads. The threads may comprise a micro-texture for improved engagement with the bone and graft.

An embodiment of the present disclosure may include a surgical screw having an outer surface, wherein the outer surface comprises a micro-texture formed thereon.

An embodiment of the present disclosure may include a surgical screw having an outer surface, wherein the outer surface comprises serrations.

An embodiment of the present disclosure may include a surgical screw having a primary thread and a set of secondary threads, wherein the set of secondary threads are disposed on the primary thread.

An embodiment of the present disclosure may include a surgical screw having a primary thread having a width and an amplitude, wherein the width increases and the amplitude decreases in the distal to proximal direction.

An embodiment of the present disclosure may include a surgical screw having a primary thread and a set of secondary threads, wherein the set of secondary threads are disposed on the peaks of the primary thread.

Those having ordinary skill in the relevant art will appreciate the advantages provide by the features of the present disclosure. For example, it is a feature of the present disclosure to provide a surgical screw with dual threads. Another feature of the present disclosure to provide such a screw with micro-texturing for preventing tendon or graft slippage. It is a further feature of the present disclosure, in accordance with one aspect thereof, to provide an injected molded surgical screw formed of PEEK with a dual thread form and micro-texturing. It is further a feature of the present disclosure to provide as surgical screw having a primary thread and a set of secondary threads.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. An apparatus for anchoring a flexible member into a bone, said apparatus comprising:
a shaft member having a proximal end and a distal end;
a primary thread disposed on the shaft member and extending from the distal end toward the proximal end; and
a set of secondary threads disposed on the primary thread.

2. The apparatus of claim 1, wherein the set of secondary threads are coextensive with the primary threads.

3. The apparatus of claim 1, wherein the set of secondary threads extends along an upper portion of the primary thread.

4. The apparatus of claim 1, wherein the set of secondary threads extends beyond the primary thread in the proximal direction along the shaft member.

5. The apparatus of claim 1, further comprising a head disposed on the proximal end of the shaft member, wherein said head comprises an engagement surface configured and dimensioned to matingly receive a drive shaft of a tool.

6. The apparatus of claim 1, wherein the primary thread has a periodicity and the set of secondary threads has a periodicity, wherein the periodicity of the primary thread and the periodicity of the secondary set of threads are substantially equal.

7. The apparatus of claim 1, wherein the primary thread comprises a continuous helical ridge curling around the shaft member, wherein the set of secondary threads is disposed on, and follows, the continuous helical ridge of the primary thread around the shaft member.

8. The apparatus of claim 7, wherein the set of secondary threads is further disposed between the curls of the continuous helical ridge.

9. The apparatus of claim 1, wherein the primary thread comprises a first portion and a second portion, the first portion and the second portion each having a terminal edge, wherein the terminal edge of the first portion is sharper than the terminal edge of the second portion.

10. The apparatus of claim 9, wherein the set of secondary threads are coextensive only with the second portion of the primary thread.

11. The apparatus of claim 1, wherein the set of secondary threads consists of a single thread.

12. The apparatus of claim 1, wherein the set of secondary threads comprises a plurality of threads.

13. The apparatus of claim 1, further comprising a microtexture on a surface of the shaft member.

14. The apparatus of claim 1, wherein the primary thread comprises a thread height and the set of secondary threads comprises a thread height, wherein the thread height of the set of secondary threads is less than 20% of the height of the primary thread.

15. The apparatus of claim 1, wherein said flexible member is human tissue.

16. A method of securing a flexible member to a bone, said method comprising:
forming a bore in the bone;
placing a portion of the flexible member into the bore;
securing the flexible member in the bore using an interference device, wherein said interference device comprises:
a shaft member having a proximal end and a distal end;
a primary thread disposed on the shaft member and extending from the distal end toward the proximal end; and
a set of secondary threads disposed on the primary thread.

17. The method of claim 16, wherein the set of secondary threads are coextensive with the primary threads.

18. The method of claim 16, wherein the set of secondary threads is disposed on an upper portion of the shaft member.

19. The method of claim 16,wherein the set of secondary threads extends beyond the primary thread toward the proximal end the shaft member.

20. The method of claim 16, wherein the primary thread has a periodicity and the set of secondary threads has a periodicity, wherein the periodicity of the primary thread and the periodicity of the secondary set of threads are substantially equal.

21. The method of claim 16, wherein the primary thread comprises a continuous helical ridge curling around the shaft member, wherein the set of secondary threads is disposed on, and follows, the continuous helical ridge of the primary thread.

22. The method of claim 21, wherein the set of secondary threads is further disposed between the curls of the continuous helical ridge.

23. The method of claim 16, wherein the primary thread comprises a thread height and the set of secondary threads comprises a thread height, wherein the thread height of the set of secondary threads is less than 20% of the height of the primary thread.

24. An apparatus for anchoring a flexible member to a bone, said apparatus comprising:
a shaft member extending between a proximal end and a distal end, the shaft member having a surface;
a primary thread disposed on the surface of the shaft; and
the primary thread having a first thread portion and a second thread portion, each of the first thread portion and the second thread portion having a terminal edge;
wherein the first thread portion extends from the distal end of the shaft member toward the proximal end of the shaft member;
wherein the second thread portion sequentially follows the first thread portion on the shaft member;
wherein the terminal edge of the first thread portion is sharper than the terminal edge of the second thread portion;
wherein the first thread portion extends between 50% and 70% of a length of the primary thread.

25. The apparatus of claim 24, wherein the length of the first thread portion is approximately 60% of the length of the primary thread.

26. The apparatus of claim 24, further comprising a transition between the first thread portion and the second thread portion, wherein the transition is abrupt.

27. The apparatus of claim 24, further comprising a head disposed on the shaft member opposite the distal end, wherein said head comprises an engagement surface configured and dimensioned to matingly receive a drive member.

28. The apparatus of claim 24, wherein the primary thread comprises a continuous helical ridge curling around the shaft member.

29. An apparatus for anchoring a flexible member into a bone, said apparatus comprising:
a shaft member extending between a proximal end and a distal end, the shaft member having a surface;
a primary thread disposed on the surface of the shaft; and
the primary thread having a thread width and a thread amplitude;
wherein the thread width increases and the thread amplitude decreases in a distal to proximal direction.

30. The apparatus of claim 29, further comprising a head disposed on the shaft member adjacent the proximal end, wherein said head comprises an engagement surface configured and dimensioned to matingly receive a drive member.

31. The apparatus of claim 29, wherein the primary thread comprises a continuous helical ridge curling around the shaft member.

32. The apparatus of claim 29, wherein the primary thread comprises a first portion adjacent the distal end of the shaft member and a second portion following the first portion, the first portion having an edge that is sharper than an edge of the second portion.

33. An apparatus for anchoring a flexible member into a bone, said apparatus comprising:
a shaft member extending between a proximal end and a distal end, the shaft member having a surface;
a primary thread disposed on the surface of the shaft, said primary thread comprising a continuous helical ridge curling around the shaft member, said ridge having a peak; and
a set of secondary threads disposed on the peak of the ridge of the primary thread.

34. The apparatus of claim 33, further comprising a head disposed on the shaft member adjacent the proximal end, wherein said head comprises an engagement surface configured and dimensioned to matingly receive a drive member.

35. The apparatus of claim 33, wherein the primary thread comprises a first portion adjacent the distal end of the shaft member and a second portion following the first portion, the first portion having an edge that is sharper than an edge of the second portion.

36. The apparatus of claim 33, wherein said flexible member is a ligament.

37. The apparatus of claim 33, wherein the set of secondary threads consists of a single thread.

38. The apparatus of claim 33, wherein the set of secondary threads comprises at least two threads.

39. The apparatus of claim 33, wherein the set of secondary threads resides only on the peak of the ridge of the primary thread, and does not reside on any sidewall or valley of the primary thread.

40. The apparatus of claim 33, wherein a number of secondary threads per peak the ridge of the primary thread increases in a distal to proximal direction.

41. An apparatus for anchoring a flexible member into a bone, said apparatus comprising:
a shaft member having a proximal end and a distal end;
a primary thread disposed on the shaft member and extending from the distal end toward the proximal end; and
a set of secondary threads disposed on the primary thread;
a head disposed on the proximal end of the shaft member, wherein said head comprises an engagement surface configured and dimensioned to matingly receive a drive shaft of a tool;
wherein the set of secondary threads extends along an upper portion of the primary thread;
wherein the set of secondary threads extends beyond the primary thread in the proximal direction along the shaft member;
wherein the primary thread has a periodicity and the set of secondary threads has a periodicity, wherein the periodicity of the primary thread and the periodicity of the secondary set of threads are substantially equal;
wherein the primary thread comprises a continuous helical ridge curling around the shaft member, wherein the set of secondary threads is disposed on, and follows, the continuous helical ridge of the primary thread around the shaft member;
wherein the set of secondary threads is further disposed between the curls of the continuous helical ridge;
wherein the primary thread comprises a first portion and a second portion, the first portion and the second portion each having a terminal edge, wherein the terminal edge of the first portion is sharper than the terminal edge of the second portion;
wherein the set of secondary threads comprises a plurality of threads;
wherein the primary thread comprises a thread height and the set of secondary threads comprises a thread height, wherein the thread height of the set of secondary threads is less than 20% of the height of the primary thread.
